(19) United States
(12) Patent Application Publication
(10) Pub. No.: US 2003/0175936 A1
(43) Pub. Date: Sep. 18, 2003

(54) Poly - gamma - glutamic acid decomposing enzyme gene and method for producing poly - gamma - glutamic acid

(75) Inventor: Yasutaka Tahara, Shizuoka-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC, Tokyo (JP)

(21) Appl. No.: 10/226,212

(22) Filed: Aug. 23, 2002

(30) Foreign Application Priority Data
Feb. 8, 2002 (JP) 2002-32837

(51) Int. Cl. C12P 21/02; C12N 1/21; C07H 21/04; C12N 9/64; C12N 15/74

(52) U.S. Cl. 435/226; 435/69.1; 435/320.1; 435/252.3; 536/23.2

(57) ABSTRACT

Poly-γ-glutamic acid is produced by culturing a microorganism belonging to the genus Bacillus which has poly-γ-glutamic acid producing ability and is modified so that an endo-type poly-γ-glutamic acid decomposing activity should be reduced or eliminated, for example, a microorganism in which expression of a novel gene coding for an endo-type poly-γ-glutamic acid decomposing enzyme is suppressed and preferably expression of a known gene coding for an exo-type poly-γ-glutamic acid decomposing enzyme (ggt) is further suppressed, in a liquid medium to produce and accumulate poly-γ-glutamic acid in a culture broth and collecting the poly-γ-glutamic acid.

### BACKGROUND OF THE INVENTION

1. Field of the Invention

The present invention relates to a novel endo-type poly-γ-glutamic acid decomposing enzyme and a gene coding for the same, as well as a microorganism belonging to the genus Bacillus in which an activity of the aforementioned enzyme is reduced or eliminated, and a method for producing poly-γ-glutamic acid using the microorganism. Polyγglutamic acid is useful in the fields of foods, cosmetics, medical products and so forth.

2. Description of the Related Art

Poly-γ-glutamic acid is known as a main substance causing stringiness of fermented soybeans (natto), and various uses thereof are expected in many fields such as those of foods, cosmetics, medical products and so forth. Poly-γ-glutamic acid is mainly produced by culturing microorganisms having poly-γ-glutamic acid producing ability, for example, strains of the genus Bacillus and collecting poly-γ-glutamic acid from the culture (refer to Gekkan Soshiki Baiyo, 16, 10, 369-372 (1990)).

As methods for increasing production amount of poly-γ-glutamic acid in microbial fermentation, there have been developed a method of culturing a mutant strain of natto-producing bacterium having poly-γ-glutamic acid producing ability and showing low ammonia productivity (Japanese Patent Laid-open Publication (Kokai) No. 8-154616), a method of culturing a microorganism having poly-γ-glutamic acid producing ability in a medium containing soy sauce koji or extract thereof, soy sauce fermentation products or a mixture thereof (Japanese Patent Laid-open Publication (Kokai) No. 8-242880), a method of culturing a mutant having poly-γ-glutamic acid producing ability of which glutamate synthase activity is deficient or reduced (Japanese Patent Laid-open Publication (Kokai) No. 2000-333690) and so forth. However, these methods suffer from a problem that, when the culture time is extended, poly-γ-glutamic acid that is once produced is degraded, resulting in reduced accumulation of poly-γ-glutamic acid.

Meanwhile, as an enzyme having an activity for decomposing poly-γ-glutamic acid, γ-glutamyltranspeptidase produced by Bacillus bacteria is known to date (Y. Ogawa, H. Hosoyama, M. Hamano & H. Motai, Agric. Biol. Chem., 55, 2971-2977 (1991); K. Xu & M. A. Strauch, J. Bacteriol., 178, 4319-4322 (1996)). However, since this enzyme is an exo-type enzyme, which successively digests poly-γ-glutamic acid from its terminus to release free glutamic acid, it has been uncertain whether this enzyme actually contributes to degradation of poly-γ-glutamic acid having a molecular weight of 1,000,000 or more. Further, as an endo-type γ-polyglutamic acid decomposing enzyme, which digests poly-γ-glutamic acid at an internal position, an enzyme produced by a microorganism belonging to the genus Myrotheclium has been reported (Japanese Patent Laid-open Publication (Kokai) No. 5-304958). However, decomposition products of poly-γ-glutamic acid produced by this enzyme are oligomers comprising 2-4 glutamic acids bonded via γ-glutamyl bonds, and therefore it has been uncertain whether this enzyme contributes to degradation of poly-γ-glutamic acid having a molecular weight of 1,000,000 or more.

Further, no endo-type poly-γ-glutamic acid decomposing enzyme produced by Bacillus bacteria is known so far, and there has been no report about production of poly-γ-glutamic acid by using a Bacillus microorganism having a suppressed poly-γ-glutamic acid decomposing enzyme activity.

### SUMMARY OF THE INVENTION

An object of the present invention is to provide a method for more efficiently producing poly-γ-glutamic acid by fermentation compared with conventional techniques.

Recently, the total genome sequence of the Bacillus subtilis 168 strain, which is widely used as an experimental microorganism, has been published (Nature, 390, 249-256 (1997)). Although any gene identified as the poly-γ-glutamic acid decomposing enzyme was not described in the table mentioned in it, the inventor of the present invention analyzed this genome sequence in detail and found that the ywtD gene, of which function was unknown, partially exhibited high homology with a known endo-type peptidase. Therefore, the inventor of the present invention estimated that the ywtD gene of Bacillus subtilis should be a novel poly-γ-glutamic acid decomposing enzyme gene and greatly affect the production of poly-γ-glutamic acid using Bacillus microorganisms, and attempted to elucidate functions of this gene. As a result, the inventor successfully demonstrated that the ywtD gene was an endo-type poly-γ-glutamic acid decomposing enzyme gene. Further, the inventor found that, in a natto-producing bacterium, Bacillus subtilis IFO16449 strain, which is poly-γ-glutamic acid producing bacterium, poly-γ-glutamic acid producing ability was markedly improved by suppressing expression of the gene and simultaneously suppressing expression of the γ-glutamyltranspeptidase gene (ggt gene), which was known to have an exo-type poly-γ-glutamic acid decomposing activity. Further, the inventor found that, also in the Bacillus subtilis UT-1 strain having improved poly-γ-glutamic acid productivity (Japanese Patent Laid-open Publication (Kokai) No. 2000-333690) by eliminating a glutamate synthase activity, poly-γ-glutamic acid producing ability was markedly improved by suppressing expression of these two poly-γ-glutamic acid decomposing enzyme genes. Thus, the inventor accomplished the present invention.

That is, the present invention provides the followings.

(1) An endo-type poly-γ-glutamic acid decomposing enzyme having the following characteristics:

1) Substrate specificity: acting on poly-γ-glutamic acid having a molecular weight of 200 kDa or more to produce poly-γ-glutamic acid having a molecular weight of 10-50 kDa

2) Optimum pH: pH 5.0

3) pH stability: stable at pH 4.0-11.0 (treated at 4° C. for 16 hours)

4) Optimum temperature: around 45° C.

5) Temperature stability: stable up to 35° C. (treated at pH 7.0 for 60 minutes)

6) Effect of addition of metal ions and inhibitor (addition of 5 mM): activated by Ba²⁺ and Mn²⁺, and its activity is inhibited by Cu²⁺ and Ni²⁺, but not affected by addition of 5 mM EDTA.

7) Molecular weight: about 46 kDa (molecular weight measured by SDS-polyacrylamide gel electrophoresis or gel filtration)

(2) The endo-type poly-γ-glutamic acid decomposing enzyme according to (1), which is a protein defined in the following (A) or (B):

(A) a protein having the amino acid sequence of SEQ ID NO: 2 shown in Sequence Listing;

(B) a protein having the amino acid sequence of SEQ ID NO: 2 shown in Sequence Listing including substitution, deletion, insertion or addition of one or several amino acids, and an endo-type poly-γ-glutamic acid decomposing enzyme activity.

(3) A DNA coding for a protein defined in the following (A) or (B):

(A) a protein having the amino acid sequence of SEQ ID NO: 2 shown in Sequence Listing;

(B) a protein having the amino acid sequence of SEQ ID NO: 2 shown in Sequence Listing including substitution, deletion, insertion or addition of one or several amino acids, and an endo-type poly-γ-glutamic acid decomposing enzyme activity.

(4) The DNA according to (3), which is defined in the following (a) or (b):

(a) a DNA which comprises the nucleotide sequence of the nucleotide numbers 41-1279 of SEQ ID NO: 1 shown in Sequence Listing;

(b) a DNA which is hybridizable with DNA having the nucleotide sequence of the nucleotide numbers 41-1279 of SEQ ID NO: 1 shown in Sequence Listing or a probe that can be prepared from the nucleotide sequence under a stringent condition, and codes for a protein having an endo-type poly-γ-glutamic acid decomposing enzyme activity.

(5) The DNA according to (4), wherein the stringent condition is a condition that washing is performed at 60° C. with salt concentrations corresponding to 1×SSC and 0.1% SDS.

(6) A microorganism belonging to the genus Bacillus, which has poly-γ-glutamic acid producing ability and is modified so that activity of the endo-type poly-γ-glutamic acid decomposing enzyme according to (1) should be reduced or eliminated.

(7) The microorganism according to (6), which is modified so that the activity of the endo-type poly-γ-glutamic acid decomposing enzyme according to (1) or (2) should be reduced or eliminated by suppressing expression of a gene coding for the enzyme.

(8) The microorganism according to (7), wherein expression of the gene coding for the endo-type poly-γ-glutamic acid decomposing enzyme according to (1) or (2) is suppressed by disrupting the gene.

(9) The microorganism according to (8), wherein the gene coding for the endo-type poly-γ-glutamic acid decomposing enzyme according to (1) or (2) is disrupted by inclusion of substitution, deletion, insertion or addition of one or several nucleotides in the nucleotide sequence of the gene.

(10) The microorganism according to (6), which is further modified so that γ-glutamyltranspeptidase activity should be reduced or eliminated.

(11) The microorganism according to (6) or (10), which is further modified so that glutamate synthase activity should be reduced or eliminated.

(12) A method for producing poly-γ-glutamic acid, which comprises culturing a microorganism according to any one of (6)-(11) in a liquid medium to produce and accumulate poly-γ-glutamic acid in a culture broth and collecting the poly-γ-glutamic acid.

The present invention provides a method for more efficiently producing poly-γ-glutamic acid by fermentation compared with conventional techniques. In particular, in preferred embodiments, produced poly-γ-glutamic acid is not degraded even after culture for a long period of time, and a marked amount thereof can be accumulated.

### BRIEF EXPLANATION OF THE DRAWINGS

FIG. 1 shows results of analyses of poly-γ-glutamic acid decomposing reactions with time in the presence of sole YwtD, sole GGT or both of YwtD and GGT by the ninhydrin method.

FIG. 2 shows results of analysis of changes in molecular weight of decomposition product with time in a poly-γ-glutamic acid decomposing reaction by YwtD.

FIG. 3 shows results of analysis of changes in molecular weight of decomposition product with time in a poly-γ-glutamic acid docomposing reaction by GGT.

FIG. 4 shows results of an analysis of changes in molecular weight of docomposition product with time in a poly-γ-glutamic acid decomposing reaction by both of YwtD and GGT.

### BEST MODE FOR CARRYING OUT THE INVENTION

Hereafter, the present invention will be explained in detail.

<1> Acquisition of DNA Fragment Including Poly-γ-Glutamic Acid Decomposing Enzyme Gene

A DNA fragment including the poly-γ-glutamic acid decomposing enzyme gene (ywtD gene) of the present invention can be obtained from an available Bacillus subtilis strain, for example, the IFO16449 strain, as follows. First, a DNA fragment including a gene homologous to the ywtD gene, of which nucleotide sequence is already known in the Bacillus subtilis 168 strain and of which function is not known, is obtained from chromosomal DNA of the Bacillus subtilis IFO16449 strain by using the polymerase chain reaction method (hereinafter, referred to as “PCR”). The obtained DNA fragment is ligated with a plasmid vector autonomously replicable within an Escherichia coli cell to produce recombinant DNA, which is then introduced into a competent cell of Escherichia coli. The obtained transformant is cultured in a liquid medium, and recombinant DNA is collected from proliferated cells. The entire nucleotide sequence of the DNA fragment included in the collected recombinant DNA is determined by the dideoxy method (refer to F. Sanger et al, Proc. Natl. Acad. Sci., 5463 (1977)), and a structure analysis of the DNA is performed to determine positions of a promoter, operator, SD sequence, initiation codon, termination codon, open reading frame and so forth.

The poly-γ-glutamic acid decomposing enzyme gene of the present invention has a sequence from GTG of the nucleotide numbers 41-43 to CAA of the nucleotide numbers 1277-1279 in the nucleotide sequence of SEQ ID NO: 1 shown in Sequence Listing. This gene codes for an endo-type poly-γ-glutamic acid decomposing enzyme having the amino acid sequence of SEQ ID NO: 2 shown in Sequence Listing.

It is sufficient that the gene of the present invention should code for the endo-type poly-γ-glutamic acid decomposing enzyme having the amino acid sequence of SEQ ID NO: 2 shown in Sequence Listing, and the nucleotide sequence is not limited to the one mentioned above. The gene may include replacement of a codon coding for each amino acid in the coding region with another equivalent codon coding for the same amino acid. Further, the gene may code for an endo-type poly-γ-glutamic acid decomposing enzyme having an amino acid sequence corresponding to the aforementioned amino acid sequence including substitution, deletion, insertion or addition of one or several amino acid residues that does not substantially impair the endo-type poly-γ-glutamic acid decomposing activity. Although the number of amino acid residues meant by the term “several” used herein may vary depending on locations of the amino acid residues in the three-dimensional structure of the endo-type poly-γ-glutamic acid decomposing enzyme or kinds of the amino acid residues, it usually means 2-200 residues, preferably 2-100 residues, more preferably 2-50 residues, most preferably 2-10 residues.

A gene coding for such an endo-type poly-γ-glutamic acid decomposing enzyme including such substitution, deletion, insertion or addition can be obtained from a variant, spontaneous mutant or artificial mutant of Bacillus subtilis or Bacillus microorganisms other than Bacillus subtilis. Further, a mutant gene coding for an endo-type poly-γ-glutamic acid including such substitution, deletion, addition or insertion can also be obtained by subjecting a gene coding for the endo-type poly-γ-glutamic acid decomposing enzyme having the amino acid sequence of SEQ ID NO: 2 to an in vitro mutagenesis treatment or site-directed mutagenesis treatment. These mutagenesis treatments can be performed by a method known to those skilled in the art as described later. A gene coding for substantially the same protein as the endo-type poly-γ-glutamic acid decomposing enzyme of the present invention can be obtained by expressing the mutant gene obtained as described above in an appropriate cell, and examining the endo-type poly-γ-glutamic acid decomposing enzyme activity of the expression product by the method described later.

Further, a gene coding for substantially the same protein as the endo-type poly-γ-glutamic acid decomposing enzyme of the present invention can also be obtained by isolating DNA which is hybridizable with DNA having the nucleotide sequence of the nucleotide numbers 41-1279 of SEQ ID NO: 1 shown in Sequence Listing or a probe that can be prepared from that nucleotide sequence under a stringent condition and codes for a protein having an endo-type poly-γ-glutamic acid decomposing enzyme activity from a mutant gene or a cell harboring such a mutant gene. The “stringent condition” referred to herein is a condition under which a so-called specific hybrid is formed. It is difficult to clearly express this condition by using numerical values since this condition depends on the GC content in each sequence or presence or absence of a repetitive sequence. However, for example, the stringent condition includes a condition under which two of DNA's having high homology, for example, two of DNA's having homology of not less than 65% are hybridized with each other, and two of DNA's having homology lower than the above are not hybridized with each other. Alternatively, the stringent condition is exemplified by a condition under which two of DNA's are hybridized with each other at salt concentrations corresponding to an ordinary condition of washing in Southern hybridization, i.e., 1×SSC, 0.1% SDS, preferably 0.1×SSC, 0.1% SDS, at 60° C.

As the probe, a partial sequence of the nucleotide sequence of SEQ ID NO: 1 can also be used. Such a probe can be prepared by PCR using oligonucleotides produced based on the nucleotide sequence of SEQ ID NO: 1 as primers and a DNA fragment including the nucleotide sequence of SEQ ID NO: 1 as a template. When a DNA fragment having a length of about 300 bp is used as a probe, a condition of 50° C., 2×SSC, 0.1% SDS can be mentioned as the condition for washing for hybridization.

Genes hybridizable under the condition described above may include those having a stop codon generated in the genes, and those having no activity due to mutation of an active center. However, such mutant genes can be easily removed by ligating the genes to a commercially available expression vector, and measuring the endo-type poly-γ-glutamic acid decomposing enzyme activity of the expression product.

The endo-type poly-γ-glutamic acid decomposing enzyme of the present invention has the following properties:

1) Substrate specificity: acting on poly-γ-glutamic acid having a molecular weight of 200 kDa or more to produce poly-γ-glutamic acid having a molecular weight of 10-50 kDa

2) Optimum pH: pH 5.0

3) pH stability: stable at pH 4.0-11.0 (treated at 4° C. for 16 hours)

4) Optimum temperature: around 45° C.

5) Temperature stability: stable up to 35° C. (treated at pH 7.0 for 60 minutes)

6) Effect of addition of metal ions and inhibitor (addition of 5 mM): activated by Ba²⁺ and Mn²⁺, and its activity is inhibited by Cu²⁺ and Ni²⁺, but not affected by addition of 5 mM EDTA

7) Molecular weight: about 46 kDa (molecular weight measured by SDS-polyacrylamide gel electrophoresis or gel filtration)

In addition to those having the amino acid sequence of SEQ ID NO: 2 shown in Sequence Listing, the endo-type poly-γ-glutamic acid decomposing enzyme of the present invention may be one having the amino acid sequence including substitution, deletion, insertion or addition of one or several amino acid residues which does not substantially impair the endo-type poly-γ-glutamic acid decomposing activity as described above.

Such an endo-type poly-γ-glutamic acid decomposing enzyme can be obtained by culturing an available Bacillus subtilis strain harboring the endo-type poly-γ-glutamic acid decomposing enzyme gene (ywtD gene) of the present invention such as the IFO16449 strain or host cells introduced with the gene in an appropriate medium and subjecting an extract or a culture broth of the obtained cells to ammonium sulfate precipitation, ethanol precipitation, anion exchange chromatography, cation exchange chromatography, hydrophobic chromatography, gel filtration chromatography and so forth, which can be appropriately combined.

<2> Construction of Bacillus Microorganism in which Expression of Poly-γ-Glutamic Acid Decomposing Enzyme Gene is Suppressed

The Bacillus microorganism of the present invention is a microorganism belonging to the genus Bacillus of which intracellular poly-γ-glutamic acid decomposing activity is reduced or eliminated. Specific examples of the Bacillus microorganism will be described later. The intracellular poly-γ-glutamic acid decomposing activity is reduced or eliminated by, for example, suppressing expression of the ywtD gene mentioned above. Further, it is preferable to simultaneously suppress expression of the ggt gene known to code for a protein exhibiting an exo-type poly-γ-glutamic acid decomposing activity. Further, the intracellular poly-γ-glutamic acid decomposing activity can also be reduced or eliminated by modifying a structure of poly-γ-glutamic acid decomposing enzyme encoded by any of these genes to reduce or eliminate its specific activity.

As means for suppressing expressions of the ywtD gene and the ggt gene, there can be mentioned, for example, a method of suppressing expressions of the genes at a transcription level by introducing substitution, deletion, insertion, addition or inversion of one or several nucleotides into promoter sequences of these genes to reduce the promoter activity (M. Rosenberg & D. Court, Ann. Rev. Genetics, 13, 319 (1979); P. Youderian, S. Bouvier & M. Susskind, Cell, 30, 843-853 (1982)). Further, expressions of these genes can be suppressed at a translation level by introducing substitution, deletion, insertion, addition or inversion of one or several nucleotides into a region between the SD sequence and the initiation codon (J. J. Dunn, E. Buzash-Pollert & F. W. Studier, Proc. Natl. Acad. Sci. U.S.A., 75, 2743 (1978)). Further, in order to reduce or eliminate the specific activity of each poly-γ-glutamic acid decomposing enzyme, there is used a method of modifying or disrupting the coding region by introducing substitution, deletion, insertion, addition or inversion of one or several nucleotides into the nucleotide sequence of the coding region of each poly-γ-glutamic acid decomposing enzyme gene. In addition to the ywtD gene and the ggt gene, the ywtD gene or the ggt gene including substitution, deletion or insertion of one or several nucleotides that does not substantially impair the encoded poly-γ-glutamic acid decomposing enzyme activity may be used as the gene into which substitution, deletion, insertion, addition or inversion of one or several nucleotides is introduced.

In order to introduce substitution, deletion, insertion, addition or inversion of a nucleotide into a gene, there can be specifically mentioned the site-directed mutagenesis (W. Kramer & H. J. Frits, Methods in Enzymology, 154, 350 (1987)) and a method comprising a treatment with a chemical agent such as sodium hyposulfite or hydroxylamine (D. Shortle & D. Nathans, Proc. Natl. Acad. Sci. U.S.A., 75, 270 (1978)). The site-directed mutagenesis is a method using a synthetic oligonucleotide, which can introduce arbitrary substitution, deletion, insertion, addition or inversion into only arbitrary limited base pairs. In order to utilize this method, a plasmid harboring a target gene that is cloned and has a determined DNA nucleotide sequence is first denatured to prepare a single strand. Then, a synthetic oligonucleotide complementary to a region where a mutation is desired to occur is synthesized. At this time, the sequence of the synthetic oligonucleotide is not made completely complementary, but is made to include substitution, deletion, insertion, addition or inversion of an arbitrary nucleotide. Thereafter, the single-stranded DNA and the synthetic oligonucleotide including substitution, deletion, insertion, addition or inversion of an arbitrary nucleotide are annealed, and further a complete double-stranded plasmid is synthesized by using Klenow fragment of DNA polymerase I and T4 ligase and introduced into competent cells of Escherichia coli. Some of the transformants obtained as described above would have a plasmid harboring a gene in which substitution, deletion, insertion, addition or inversion of an arbitrary nucleotide is fixed. As a similar method that enables introduction of mutation and thereby modification or disruption of the gene, there can be mentioned the recombinant PCR method (PCR Technology, Stockton press (1989)).

Further, the method using a treatment with a chemical agent is a method of randomly introducing a mutation including substitution, deletion, insertion, addition or inversion of nucleotide into a DNA fragment including a target gene by directly treating the DNA fragment with sodium hyposulfite, hydroxylamine or the like.

Expression of the ywtD gene and the ggt gene in a cell can be suppressed by substituting the gene obtained as described above, which is modified or disrupted by introducing a mutation, for a normal gene on the chromosome of a Bacillus microorganism. As a method for gene substitution, there can be mentioned a method utilizing homologous recombination (Experiments in Molecular Genetics, Cold Spring Harbor Laboratory press (1972); S. Matsuyama & S. Mizushima, J. Bacteriol., 162, 1196 (1985)). Ability to cause homologous recombination is a property generally possessed by Bacillus microorganisms. When a plasmid harboring a sequence having homology to a sequence on the chromosome or the like is introduced into a bacterial cell, recombination occurs at a site of the sequence having homology in a certain frequency. As a result, there can be obtained a strain in which the gene introduced with a mutation is fixed on the chromosome and substitute for the original normal gene. By selecting such a strain, there can be obtained a strain in which a gene modified or disrupted by introducing a mutation including substitution, deletion, insertion, addition or inversion of a nucleotide substitutes for a normal gene on the chromosome.

A Bacillus microorganism used for the gene substitution is a microorganism having poly-γ-glutamic acid producing ability. As Bacillus microorganisms having poly-γ-glutamic acid producing ability, there can be mentioned, for example, Bacillus subtilis, Bacillus licheniformis, Bacillus anthracis, Bacillus megaterium and so forth. More specifically, there can be mentioned, for example, Bacillus subtilis IFO3335, Bacillus subtilis IFO3336 (M. Kunioka and A. Goto, Appl. Microbiol. Biotecnol., 40, 867-872 (1994)), Bacillus subtilis IFO16449, Bacillus licheniformis ATCC9945 (F. A. Troy, J. Biol. Chem., 248, 305-315 (1973)) and so forth, and commercially available natto-producing bacteria usually used to produce natto (fermented soybeans) such as Miyagino, Takahashi, Asahikawa, Matsumura and Naruse natto-producing bacteria. Further, expression of the poly-γ-glutamic acid decomposing enzyme gene may be suppressed in a Bacillus microorganism having improved poly-γ-glutamic acid producing ability obtained from any of these bacterial strains by breeding. The Bacillus microorganism having improved poly-γ-glutamic acid producing ability can be obtained by, for example, suppressing expression of glutamate synthase gene (gltA gene) by disrupting the gene (Japanese Patent Laid-open Publication (Kokai) No. 2000-333690).

As other methods for introducing a mutation into the ywtD gene or the ggt gene to modify or disrupt the gene, there can also be mentioned a method of subjecting a Bacillus microorganism cell having the gene to a treatment using a chemical agent such as N-methyl-N′-nitro-N-nitrosoguanidine or nitrous acid or irradiation of ultraviolet rays, radiation or the like to cause a genetic mutation.

In the examples described later, a Bacillus subtilis strain in which a function of the poly-γ-glutamic acid decomposing enzyme gene was eliminated was created by substituting a poly-γ-glutamic acid decomposing enzyme gene in which a part of the coding region was deleted and which was inserted with a drug resistance gene instead for a poly-γ-glutamic acid decomposing enzyme gene on the chromosome of Bacillus subtilis using the aforementioned method utilizing homologous recombination.

In one bacterial strain, expression of only the ywtD gene of the present invention may be suppressed, or expressions of both the ywtD gene and the ggt gene may also be suppressed. In the present invention, a bacterial strain in which expressions of both the ywtD gene and the ggt gene are suppressed is preferred. Further, a bacterial strain in which expression of the gltA gene is suppressed in addition to these genes is more preferred.

<3>Production of Poly-γ-Glutamic Acid by Using Bacillus Microorganism in which Expression of Poly-γ-Glutamic Acid Decomposing Enzyme Gene is Suppressed

By culturing a Bacillus microorganism obtained as described above, in which expression of the poly-γ-glutamic acid decomposing enzyme gene is suppressed, a marked amount of poly-γ-glutamic acid is produced and accumulated in a culture broth. Although the accumulation amount of poly-γ-glutamic acid is increased only by suppressing expression of the ggt gene, which is known to code for a protein having a poly-γ-glutamic acid decomposing activity, suppression of the expression of poly-γ-glutamic acid decomposing enzyme gene of the present invention is more effective for improvement of the poly-γ-glutamic acid accumulation amount, and a favorable result for production of poly-γ-glutamic acid is obtained by using a bacterial strain in which expressions of both the ggt gene and the poly-γ-glutamic acid decomposing enzyme gene of the present invention are suppressed.

A medium used to produce poly-γ-glutamic acid is a usual medium containing a carbon source, nitrogen source, inorganic ions and other organic trace nutrient sources as required, but it is particularly preferable to add a medium with glutamic acid or a salt thereof, for example, sodium glutamate, potassium glutamate or the like, since it provides efficient production of poly-γ-glutamic acid. As specific examples of medium ingredients, appropriate combinations of the followings are used.

First, examples of the carbon source include glucose, fructose, sucrose, maltose, raw sugars, molasses (for example, beet molasses, sweet potato molasses), various starches (for example, tapioca, sago, sweet potato, potato, maize) or saccharified solutions thereof obtained by using acids or enzymes and so forth or combinations of two or more of them.

Further, examples of the nitrogen source include glutamic acid, sodium glutamate, potassium glutamate, soy sauce koji or an extract thereof, soy sauce fermentation products such as soy sauce and sediment of soy sauce or a mixture thereof, organic nitrogen sources such as peptone, soybean meal, corn steep liquor, yeast extract, meat extract, soybean itself or defatted soybean, powder, grain or extract thereof and urea, inorganic nitrogen sources such as ammonium salts of sulfuric acid, nitric acid, hydrochloric acid, carbonic acid and so forth, ammonia gas and aqueous ammonia, appropriate combinations of two or more kinds thereof and so forth.

Further, in addition to the aforementioned carbon sources and nitrogen sources, there can be used various inorganic salts necessary for growth of microorganisms, for example, sulfates, hydrochlorides, phosphates and acetates of calcium, potassium, sodium, magnesium, manganese, iron, copper, zinc etc., amino acids, vitamins and so forth. As the amino acids, in addition to the aforementioned glutamic acid, aspartic acid, alanine, leucine, phenylalanine, histidine etc. can be used as required. As the vitamins, biotin, thiamine etc. can be used.

Further, as a medium material for a solid culture, for example, cooked soybeans, barley, wheat, buckwheat, maize or a mixture thereof, or any of these added with glutamic acid or a metal salt thereof can be preferably used.

In order to culture a Bacillus microorganism in which expression of the poly-γ-glutamic acid decomposing enzyme gene is suppressed, the aforementioned medium is sterilized by a usual method, for example, at 110-140° C. for 8-15 minutes, and added with the microorganism. In the case of liquid culture, culture is preferably performed under an aerobic condition as in culture with shaking, culture with aeration and stirring or the like. For such culture, culture temperature is 25-50° C., preferably 37-42° C.

Further, pH of the medium is adjusted by using sodium hydroxide, potassium hydroxide, ammonia, an aqueous solution thereof or the like, and it is desired that culture should be performed at pH 5-9, preferably pH 6-8.

Further, culture period may be usually about 2-4 days. Further, also in the case of solid culture, a culture temperature of 25-50° C., preferably 37-42° C., and pH of 5-9, preferably 6-8, are employed during the culture as in the case of the liquid culture. When the microorganism is cultured as described above, poly-γ-glutamic acid is accumulated mainly outside the cells and contained in the culture.

In order to separate and collect poly-γ-glutamic acid from this culture, there can be used known methods, for example, (1) a method of extracting and isolating poly-γ-glutamic acid from solid culture by using saline at a concentration of 20% or lower (Japanese Patent Laid-open Publication (Kokai) No. 3-30648), (2) a precipitation method using copper sulfate (B. C. Throne, C. C. Gomez, N. E. Noues and R. D. Housevright, J. Bacteriol., 68, 307 (1954)), (3) an alcohol precipitation method (R. M. Vard, R. F. Anderson and F. K. Dean, Biotechnology and Bioengineering, 5, 41 (1963); S. Sawa, T. Murakawa, S. Murao, S. Omata, Noka (Journal of Japan Society for Bioscience, Biotechnology and Agrochemistry), 47, 159-165 (1973); H. Fujii, Noka, 37, 407-412 (1963) etc.), (4) a chromatography method using a crosslinked chitosan mold product as an adsorbent (Japanese Patent Laid-open Publication (Kokai) No. 3-244392), (5) a molecular ultrafiltration method using a molecular ultrafiltration membrane, (6) a method consisting of an appropriate combination of the aforementioned (1)-(5) and so forth. The poly-γ-glutamic acid isolated and collected as described above may be made into a solution or powder containing poly-γ-glutamic acid as required by known techniques such as concentration, hot-air drying and lyophilization in a conventional manner.

### EXAMPLES

The present invention will be explained more specifically with reference to the following examples. However, the scope of the present invention is not limited to these examples.

### Example 1

Cloning of ywtD Gene of Bacillus subtilis IFO16449 Strain and Purification and Property Examination of Gene Product

(1) Acquisition of ywtD Gene of Bacillus subtilis IFO16449 Strain

The following primers were synthesized based on the sequence of the ywtD gene in the data bank of Bacillus subtilis.

| | |
|---|---|
| (Forward) | (SEQ ID NO: 3) |

5′-GGA TCC GTT AAA ACT GCA AAA AGA GG

| | |
|---|---|
| (Reverse) | (SEQ ID NO: 4) |

5′-TTT CTC GAG TTG CAC CCG TAT ACT TC

An about 1.3-kbp ywtD gene fragment was amplified by PCR using the above primers and chromosomal DNA of the Bacillus subtilis IFO16449 strain as a template. This fragment was digested with restriction enzymes BamHI and XhoI (Takara Shuzo) and subjected to electrophoresis on 1% agarose gel to collect an amplified fragment. This 1.3-kb fragment was inserted between the BamHI and XhoI sites of pET23a (+) (Novagen) to prepare an expression plasmid (hereinafter, referred to as “pNDH”) for expression of the fragment as a protein including a histidine tag fused to the C-terminus.

The result of sequencing of the nucleotide sequence of the inserted fragment in pNDH is shown as SEQ ID NO: 1 in Sequence Listing. The amino acid sequence of the endo-type poly-γ-glutamic acid decomposing enzyme encoded by the ywtD gene in this gene fragment is shown as SEQ ID NO: 2 in Sequence Listing. The sequence of the ywtD gene derived from the Bacillus subtilis IFO16449 strain showed homology of 99% to the sequence of the ywtD gene of the Bacillus subtilis 168 strain (Nature, 390, 249-256 (1997), SubtiList database accession number BG12535) at the amino acid sequence level.

(2) Expression and Purification of ywtD Gene Product of Bacillus subtilis IFO16449 Strain

Subsequently, a ywtD gene product having the histidine tag fused to the C terminus (H-Ywt) was expressed in Escherichia coil in a large amount to prepare a purified enzyme preparation. The Escherichia coli (E. coli) BL21(DE3) strain (Novagen) was transformed with the plasmid pNDH to obtain an E. coli BL21/pNDH strain harboring the plasmid pNDH. This strain was inoculated into 100 ml of LB medium (1% polypeptone, 0.5% yeast extract, 1% NaCl, pH 7.0) containing 50 μg/ml of ampicillin and cultured at 37° C. When the bacteria proliferated to such an extent that the absorbance at 660 nm should reach 0.5, 0.4 mM IPTG (isopropyl β-D-thiogalactopyranoside, Takara Shuzo) was added to the culture and culture was further continued for 5 hours.

The cells collected from the culture broth by centrifugation were suspended in 5 ml of 50 mM phosphate buffer (pH 7.0) and disrupted by ultrasonication at 4° C. for 5 minutes. Insoluble fractions were removed by centrifugation to prepare a cell-free extract. The extract was adsorbed on a HiTrap Chelating Sepharose column (1 ml of carrier, Amersham Pharmacia Biotech) equilibrated with 20 mM phosphate buffer (pH 7.5) containing 500 mM NaCl and 10 mM imidazole. The column was washed with the same buffer, and then the enzyme was eluted stepwise with 20 mM phosphate buffer (pH 7.5) containing 500 mM NaCl and 50 mM or 100 mM imidazole.

Active fractions were collected, concentrated by ultrafiltration, loaded on a Sephacryl S-200 column (1.5×60 cm, Amersham Pharmacia Biotech) equilibrated with 50 mM phosphate buffer (pH 7.0) containing 150 mM NaCl and eluted with the same buffer at a flow rate of 0.5/min. As a result of the above operations, 0.5 mg of purified H-YwtD protein could be prepared. This enzyme preparation was uniform as determined by SDS-polyacrylamide electrophoresis.

(3) Property Examination of ywtD Gene Product Derived from Bacillus subtilis IFO16449 Strain

Property examination of the enzyme was performed by using the purified enzyme preparation. As a substrate for the reaction, there was used poly-γ-glutamic acid having an average molecular weight of 50 kDa or more prepared from a culture broth of the Bacillus subtilis IFO16449 strain according to the method of Kuninaka et al. (Biosci. Biotech. Biochem., 56, 1031-1035 (1992)).

The poly-γ-glutamic acid decomposing activity was measured under the following conditions. The reaction was performed in a reaction mixture (1 ml) containing 4 mg/ml of poly-γ-glutamic acid (having an average molecular weight of 500,000 or more), potassium phosphate buffer (pH 7.0) and the enzyme at pH 7.0 and 37° C. The reaction was terminated with 1.2 ml of a methyl cellosolve solution for ninhydrin reaction, and free glutamic acid was quantified by the ninhydrin method. The amount of enzyme required to produce 1 μmol of L-glutamic acid in 1 minute under these standard reaction conditions was defined as 1 unit. The specific activity was determined as number of the unit per 1 mg of the protein. The protein was quantified according to the method of Horio et al. (Basic Experimental Methods for Protein and Enzyme, Second Revised Edition, Konando (1994)). That is, Solution A (0.1 N NaOH solution containing 2% Na₂CO₃) and Solution B (1% aqueous sodium citrate solution containing 0.5% CUSO₄) were mixed at a ratio of 50:1 to prepare Solution C. In an amount of 0.3 ml of a sample solution diluted to a protein concentration of 50-300 μg/ml and 3 ml of Solution C were mixed and left standing at room temperature for 20 minutes. The mixture was added with 0.3 ml of a phenol reagent (Wako Pure Chemical Industries) diluted two-fold and left standing for 30 minutes, and the absorbance at 750 nm was measured. The calibration curve was prepared by using 0-400 μg/ml of bovine serum albumin (Wako Pure Chemical Industries).

The molecular weight of the poly-γ-glutamic acid decomposition product was analyzed by HPLC under the following conditions. The molecular weight was calculated by using poly-α-glutamic acid samples having known molecular weights (molecular weights of 14 kDa, 32 kDa and 58 kDa, Sigma) and Pullulan (molecular weight of 200 kDa, Tokyo Kasei Kogyo) as standards.

Column: Asahipak GF-7M HQ (7.6×300 mm, Showa Denko)

Mobile phase: 50 mM phosphate buffer (pH 6.8)

Flow rate: 0.6 ml/min

Temperature: 32° C.

Detection: RI Detector

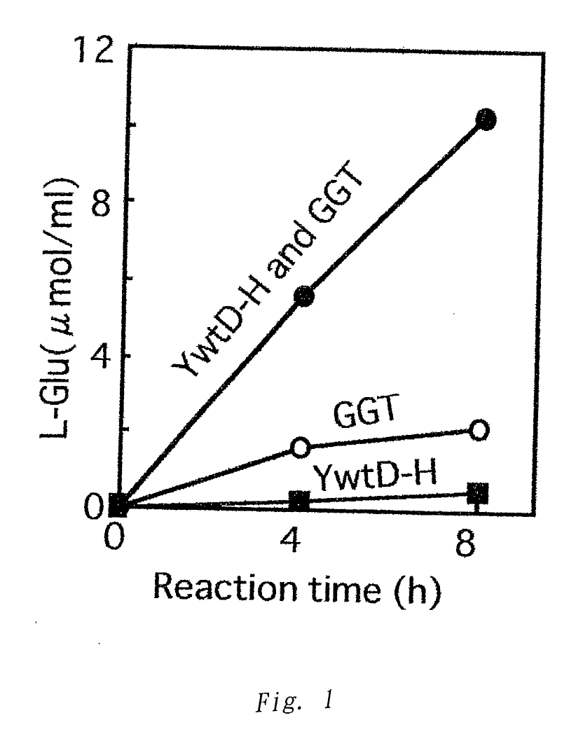

First, the poly-γ-glutamic acid decomposing activity of the purified enzyme was measured by the ninhydrin method. When the reaction was performed by adding 90 μg of the purified enzyme under the standard conditions, glutamic acid produced by the degradation of poly-γ-glutamic acid increasing with the reaction time was detected, and thus it was shown that the enzyme had the poly-γ-glutamic acid decomposing activity (FIG. 1, ▪). After the reaction for 2 hours, 0.10 μmol of L-glutamic acid was detected, and the specific activity of the purified preparation was calculated to be 9.26 mU/mg protein in this method.

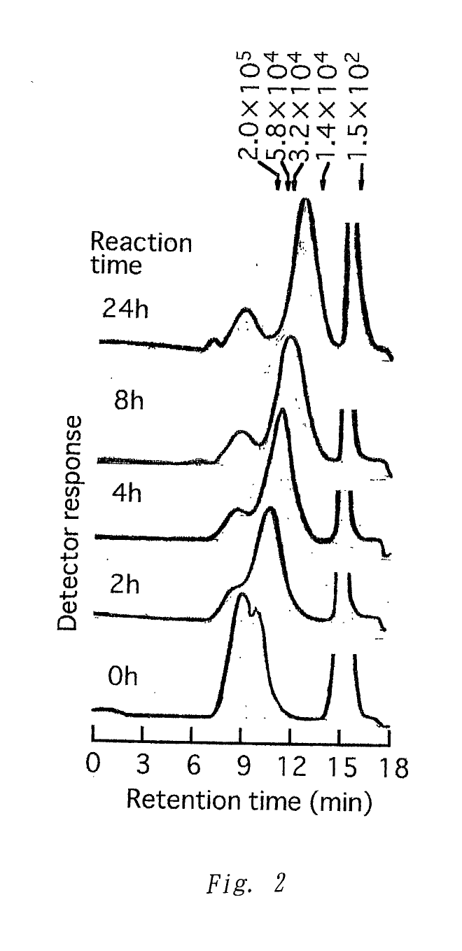

The results of the molecular weight measurement of the degraded product by HPLC are shown in FIG. 2. There was confirmed decrease of the molecular weight of poly-γ-glutamic having a molecular weight of 200 kDa or more with progress of the reaction. After the reaction for 24 hours, poly-γ-glutamic acid was degraded into products having a molecular weight of 10-50 kDa.

The above results revealed that the enzyme had an activity for decomposing poly-γ-glutamic acid as an endo-type enzyme.

The properties of the enzyme were further examined in detail, and it was found that the enzyme had the following characteristics.

1) Substrate specificity: the enzyme acted on poly-γ-glutamic acid having a molecular weight of 200 kDa or more to produce poly-γ-glutamic acid having a molecular weight of 10-50 kDa.

2) Optimum pH: pH 5.0

3) pH stability: stable at pH 4.0-11.0 (treated at 4° C. for 16 hours)

4) Optimum temperature: around 45° C.

5) Temperature stability: stable up to 35° C. (treated at pH 7.0 for 60 minutes)

6) Effect of addition of metal ions and inhibitor (addition of 5 mM): the enzymatic activity was promoted by 43% with addition of Ba²⁺ and by 10% with addition of Mn²⁺. On the other hand, 100% activity inhibition was observed with addition of Cu²⁺, and 84% inhibition was observed with addition of Ni²⁺. The activity was not affected by addition of 5 mM EDTA.

7) Subunit molecular weight: calculated as about 46 kDa based on results of SDS-polyacrylamide gel electrophoresis under a reducing condition.

8) Molecular weight: calculated as 46 kDa based on results of FPLC (Sephacryl S-200, Amersham Pharmacia Biotech).

From the results of the above 7) and 8), the enzyme was estimated to be a monomer.

### Example 2

Decomposition of Poly-γ-Glutamic Acid in the Presence of Both of Poly-γ-Glutamic Acid Decomposing Enzyme (YwtD) and γ-Glutamyltransferase (GGT)

(1) Partial Purification of γ-Glutamyltranspeptidase (GGT) Derived from Bacillus subtilis IFO16449 Strain

The Bacillus subtilis IFO16449 strain was inoculated into 100 ml of SY medium (5% sucrose, 2% yeast extract, 0.25% KH₂PO₄, 0.05% MgSO4.7H₂O, 0.25% NaCl, pH 7.0) and cultured at 37° C. for 15 hours. The cells were removed by centrifugation, and γ-glutamyltranspeptidase (GGT) was partially purified from the culture supernatant by PMSF (phenylmethane sulfonylfluoride) treatment and DEAE column chromatography according to the method of Ogawa et al. (Y. Ogawa, H. Hosoyama, M. Hamano, H. Motai, Agric. Biol. Chem., 55, 2971-2977 (1991)) to prepare about 15 mg of partially purified GGT.

(2) Decomposing Reaction of Poly-γ-Glutamic Acid by GGT Derived from Bacillus subtilis IFO16449 Strain

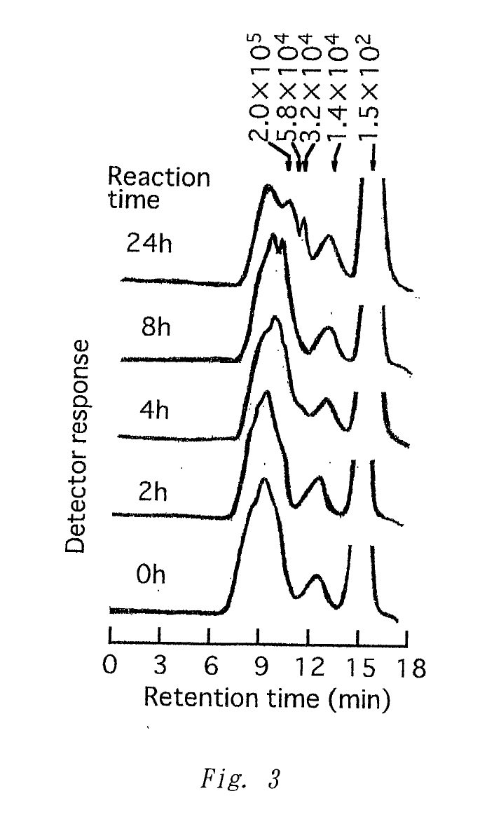

A decomposing reaction of poly-γ-glutamic acid was performed in the same manner as in Example 1 by using the partially purified GGT. When the poly-γ-glutamic acid decomposing activity was measured, glutamic acid produced by the degradation of poly-γ-glutamic acid increasing with the reaction time was detected (FIG. 1, ◯). When the reaction was performed under the standard conditions with addition of 250 μg of the partially purified GGT, 1.3 μmol of L-glutamic acid was detected after the reaction for 2 hours, and the specific activity of the purified preparation was calculated to be 43.3 mU/mg protein by the ninhydrin method. The results of the molecular weight measurement of the decomposition products by HPLC are shown in FIG. 3. When the reaction was performed by using GGT, almost no decrease in the molecular weight of poly-γ-glutamic acid was observed even after the reaction for 24 hours.

(3) Decomposing Reaction of Poly-γ-Glutamic Acid in the Presence of Both of YwtD-H Derived from Bacillus subtilis IFO16449 Strain and GGT

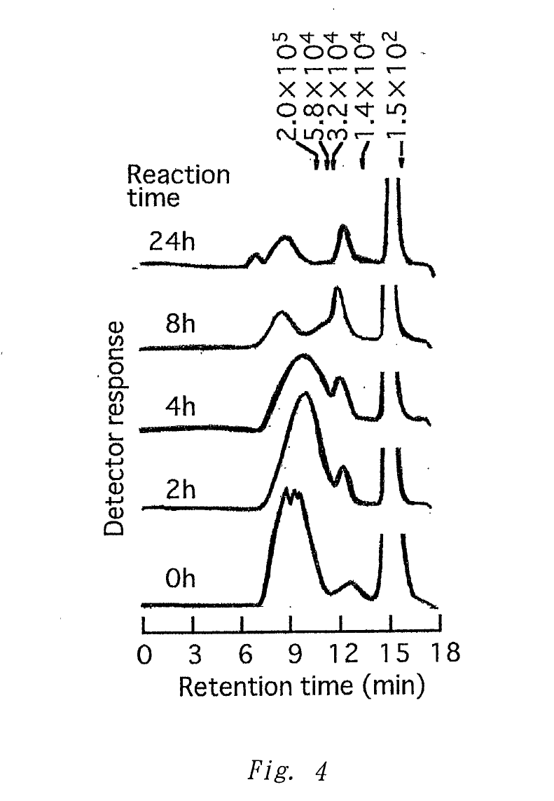

Subsequently, 90 μg of Ywt-H prepared in Example 1 and 250 μg of the partially purified GGT were simultaneously added to perform a decomposing reaction of poly-γ-glutamic acid. When the poly-γ-glutamic acid decomposing activity was measured, glutamic acid produced by the degradation of poly-γ-glutamic acid increasing with the reaction time was detected. As shown in FIG. 1, the amount of the produced glutamic acid markedly increased in comparison with the case where each enzyme was solely added (FIG. 1, ●). The results of the molecular weight measurement of the decomposition product obtained by simultaneously adding two kinds of the enzymes are shown in FIG. 4. In the case of the decomposing reaction using GGT solely, almost no decrease in the molecular weight of poly-γ-glutamic acid was observed. In the case of the decomposing reaction using Ywt-H solely, only poly-γ-glutamic acid having a molecular weight of 10-50 kDa or less was degraded. On the other hand, when these two kinds of enzymes were simultaneously added to perform the reaction, poly-γ-glutamic acid having a molecular weight of 200 kDa or more was almost completely degraded into those having a lower molecular weight after the reaction for 24 hours.

These results strongly suggested that poly-γ-glutamic acid should be first degraded by the ywtD gene product as an endo-type enzyme, and then the produced low molecular weight poly-γ-glutamic acid should be degraded by GGT as an exo-type enzyme into oligomers and monomers of glutamic acid. Thus, it was considered that the presence of these two kinds of enzymes greatly affected production of poly-γ-glutamic acid by fermentation using Bacillus microorganisms.

### Example 3

Construction of Bacillus subtilis IFO16449 Strain of Which ywtD Gene is Disrupted

A 1.3-kbp fragment including the ywtD gene of the Bacillus subtilis IFO16449 strain was amplified by PCR in the same manner as in Example 1, and the amplification product was inserted into the HincII site of pUC19 (Takara Shuzo) to construct a plasmid pUCywtD. Subsequently, a 1.5-kbp erythromycin resistance gene (erm) fragment excised from pMUTin4MCS (obtained from Bacillus Genetic Stock Center) with a restriction enzyme AccII (Takara Shuzo) was inserted into the BglI site of pUCywtD to construct a plasmid pUCΔywtD for disruption of the ywtD gene.

The Bacillus subtilis IFO16449 strain was inoculated into 20 ml of 2×TY medium (1.6% polypeptone, 1% yeast extract, 0.5% NaCl, pH 7) and cultured overnight at 37+ C. and 200 rpm. In an amount of 200 μl of this culture broth was inoculated into 20 ml of SPI medium (0.6% KH₂PO₄, 1.4% K₂HPO₄, 0.2% ammonium sulfate, 0.1% sodium citrate, 0.02% magnesium sulfate, 0.5% glucose, 0.02% casamino acid, 0.1% yeast extract, 50 mg/ml tryptophan, 50 mg/ml leucine), and the strain was cultured until a later stage of logarithmic growth phase. Further, 10 ml of this culture broth was inoculated into 100 ml of SPII medium (0.6% KH₂PO₄, 1.4% K₂HPO₄, 0.2% ammonium sulfate, 0.1% sodium citrate, 0.02% magnesium sulfate, 0.5% glucose, 75 mg/ml CaCl₂, 508 mg/ml MgCl₂), and the strain was cultured at 37° C. with shaking at 200 rpm for 90 minutes to prepare competent cells.

In an amount of 10 ml of a culture broth of the prepared competent cells was placed in a 20-ml conical flask and added with 10 μg of the previously constructed plasmid pUCΔywtD for gene disruption, and the cells were cultured at 37° C. and 120 rpm for 60 minutes. In an amount of 100 μl of the culture broth was plated on a 2×YT plate containing 4 μg/ml of erythromycin (Em), and the cells were cultured at 37° C. for about 15 hours as standing culture to obtain a grown transformant strain (ywtD::erm strain). It was confirmed by Southern blot hybridization that the ywtD gene of the ywtD::erm strain was disrupted by insertion of the erythromycin resistance gene into the ywtD gene.

### Example 4

Production of Poly-γ-Glutamic Acid by Bacillus subtilis IFO16449 Strain of Which ywtD Gene is Disrupted

The Bacillus subtilis IFO16449 strain and the strain obtained in Example 3 of which ywtD gene was disrupted were each inoculated into 30 ml of γ-polyglutamic acid production medium (2% glutamic acid, 1% ammonium sulfate, 0.1% Na₂HPO₄, 0.1% KH₂PO₄, 0.05% MgSO₄, 0.02% CaCl₂, 0.005% FeCl₃, 0.002% MnCl₂, 0.5 μg/ml biotin, pH 7.5) contained in a 200-ml ribbed conical flask and cultured at 37° C. with shaking at 170 rpm for 48 hours. For the culture of the strain of which ywtD gene was disrupted, 1 μg/ml of erythromycin was added.

After completion of the culture, the liquid culture was diluted 5-fold, and the poly-γ-glutamic acid produced in the culture broth was quantified. The poly-γ-glutamic acid was quantified by the safranine method according to the procedure of Bovarnick et al. (M. Bovernick, F. Eisenberg, D. O' Connell, J. Victor & P. Owases, J. Biol. Chem., 593 (1954)).

As a result of the measurement of the amount of poly-γ-glutamic acid in the culture broth, it was found that the produced amount was 4.8 mg/ml for the IFO16449 strain, which was a parent strain, but the produced amount was 5.4 mg/ml for the ywtD gene disrupted strain, which corresponded to an increase of the amount of the produced poly-γ-glutamic acid by 1.12 times in comparison with the parent strain. The results revealed that activity of the poly-γ-glutamic acid decomposing enzyme encoded by the ywtD gene greatly affected production of poly-γ-glutamic acid.

### Example 5

Bacillus subtilis IFO16449 Strain of Which ggt Gene is Disrupted and Bacillus subtilis IFO16449 Strain of Which ywtD and ggt Genes are Both Disrupted

According to the method of Ogawa et al. (Y. Ogawa, D. Sugiura, H. Motai, K. Yuasa & Y. Tahara, Biosci. Biotech. Biochem., 61, 1596-1600 (1997)), the ggt gene of the Bacillus subtilis IFO16449 strain was cloned into pUC18 (Takara Shuzo) to construct a plasmid pGT2. Subsequently, a 1.0-kbp chloramphenicol resistance gene (cat) fragment excised from pC194 (obtained from Bacillus Genetic Stock Center) with restriction enzymes NaeI and XhoII (Takara Shuzo) was inserted into the BstPI site of pGT2 to construct a plasmid pUCΔggt for disruption of the γ-glutamyltranspeptidase gene (ggt gene).

The Bacillus subtilis IFO16449 strain was transformed with the plasmid pUCΔggt for disruption of the ggt gene in the same manner as in Example 3 to disrupt the ggt gene by homologous recombination. A culture broth of cells introduced with the plasmid was plated on a 2×YT plate containing 5 μg/ml of chloramphenicol (Cm), and the cells were cultured as standing culture at 37° C. for about 15 hours to obtain a grown transformant strain (ggt::cat strain). The γ-glutamyltranspeptidase activity of the ggt::cat strain was measured according to the method of Ogawa et al. (Y. Ogawa, H. Hosoyama, M. Hamano & H. Motai, Agric. Biol. Chem., 55, 2971-2977 (1991)) to confirm GGT deficiency.

Subsequently, by using the plasmid pUCΔywtD for disruption of the ywtD gene used in Example 3, the ywtD gene of the ggt::cat strain was disrupted in the same manner. A culture broth of the cells introduced with the plasmid was plated on a 2×YT plate containing 5 μg/ml of chloramphenicol (Cm) and 1 μg/ml of erythromycin, and the cells were cultured as standing culture at 37° C. for about 15 hours to obtain a grown transformant strain (ggt::cat+ywtD::emr strain). Disruption of ywtD was confirmed by Southern hybridization.

### Example 6

Production of Poly-γ-Glutamic Acid by Strain of Which ggt Gene Disrupted and Strain of Which ywtD and ggt Genes are Both Disrupted

The Bacillus subtilis IFO16449 strain, the ggt gene disrupted strain and the strain of which ywtD and ggt genes were both disrupted, which were obtained in Example 5, were each inoculated into 30 ml of γ-polyglutamic acid production medium (2% glutamic acid, 1% ammonium sulfate, 0.1% Na₂HPO₄, 0.1% KH₂PO₄, 0.05% MgSO₄, 0.02% CaCl₂, 0.005% FeCl₃, 0.002% MnCl₂, 0.5 μg/ml biotin, pH 7.5) contained in a 200-ml ribbed conical flask and cultured at 37° C. with shaking at 170 rpm for 48 hours. For the culture of the ggt gene disrupted strain, 5 μg/ml of chloramphenicol was added. For the culture of the strain of which ywtD and ggt genes were both disrupted, 1 μg/ml of erythromycin and 5 μg/ml of chloramphenicol were added.

After completion of the culture, poly-γ-glutamic acid produced in the culture broth was measured in the same manner as in Example 4. As a result, the produced amount was 4.8 mg/ml for the IFO16449 strain, which was a parent strain. For the strain of which ggt gene was solely disrupted, productivity was improved about 1.20 times, and the produced amount was 5.8 mg/ml. For the strain of which ywtD and ggt genes were both disrupted, productivity was further improved in comparison with the strains in which each gene was solely disrupted, with a productivity 1.31 times higher than that of the parent strain, and 6.3 mg/ml of poly-γ-glutamic acid was accumulated. This results revealed that activity of poly-γ-glutamic acid decomposing enzyme encoded by the ywtD gene and activity of γ-glutamyltranspeptidase encoded by the ggt gene greatly affected production of poly-γ-glutamic acid.

### Example 7

Production of Bacillus subtilis IFO16449 Strain of Which ywtD ggt and gltA Genes are All Disrupted

The following primers were synthesized based on the nucleotide sequence of glutamate synthase gene (gltA gene) in the data bank of Bacillus subtilis.

| | |
|---|---|
| (Forward) | |
| 5′-CTT GGA TGG AGA ACT GTA CCT G | (SEQ ID NO: 5) |
| (Reverse) | |
| 5′-GGC GCT GAA ATT AGG TGC TG | (SEQ ID NO: 6) |

About 6-kbp gltA gene fragment was prepared by PCR using these primers and chromosomal DNA of the Bacillus subtilis IFO16449 strain as a template. This fragment was digested with a restriction enzyme EcoT22I (Takara Shuzo), and the obtained 5-kb fragment was inserted into the PstI site of pUC19 (Takara Shuzo) to construct a plasmid pGO. Subsequently, a 0.9-kbp neomycin resistance gene (neo) fragment excised from a cosmid Lorist6 (Nippon Gene) with restriction enzymes SalI and BclI (Takara Shuzo) was inserted into the NaeI site of the plasmid pGO to construct a plasmid pGOCM for disruption of the gltA gene.

The Bacillus subtilis IFO16449 strain and the strain constructed in Example 4 of which ywtD and ggt genes were both disrupted (ggt::cat +ywtD::emr strain) were transformed with the plasmid pGOCM for disruption of the gltA gene in the same manner as in Example 3 to disrupt the gltA gene by homologous recombination. In an amount of 100 μl of a culture broth of the cells introduced with the plasmid was plated on a 2×YT plate containing 5 μg/ml of neomycin (Nm), or 5 μg/ml of neomycin, 5 μg/ml chloramphenicol and 1 μg/ml erythromycin, and the cells were cultured as standing culture at 37° C. for about 15 hours to obtain grown transformant strains (gltA::neo strain and ggt::cat+ywtD::emr+gltA::neo strain).

### Example 8

Production of Poly-γ-Glutamic Acid by Strain of Which ywtD. ggt and gltA Genes are All Disrupted

The Bacillus subtilis IFO16449 strain, the gltA gene disrupted strain obtained in Example 6 and the strain of which ywtD, ggt and gltA genes were all disrupted were inoculated into 30 ml of γ-polyglutamic acid production medium (2% glutamic acid, 1% ammonium sulfate, 0.1% Na₂HPO₄, 0.1% KH₂PO₄, 0.05% MgSO₄, 0.02% CaCl₂, 0.005% FeCl₃, 0.002% MnCl₂, 0.5 μg/ml biotin, pH 7.5) contained in a 200-ml ribbed conical flask and cultured at 37° C. with shaking at 170 rpm for 96 hours. For the culture of the gltA gene disrupted strain, 5 μg/ml of neomycin was added. For the culture of the strain of which ywtD, ggt and gltA genes were all disrupted, 1 μg/ml of erythromycin, 5 μg/ml of chloramphenicol and 5 μg/ml of neomycin were added.

The amounts of poly-γ-glutamic acid in the culture broth at 48 hours and 96 hours after the start of the culture were measured in the same manner as described in Example 3, and the results are shown in Table 1.

TABLE 1
Amount of poly-γ-glutamic acid produced
by each bacterial strain (mg/ml)

Culture time

| Bacterial strain | 48 h | 96 h |
|---|---|---|
| IFO16449 strain | 8.09 | 6.65 |
| Strain of which gltA | 11.90 | 9.45 |
| gene was disrupted | | |
| Strain of which ywtD, | 12.77 | 16.10 |
| ggt and gltA genes were | | |
| all disrupted | | |

After the culture for 48 hours, productivity was significantly improved in the gltA gene disrupted strain in comparison with its parent strain, the IFO16449 strain, as shown in Japanese Patent Laid-open Publication (Kokai) No. 2000-333690. However, even when the culture time was extended, the amount of the accumulated poly-γ-glutamic acid did not increase, but rather the accumulated amount decreased due to the degradation of the produced poly-γ-glutamic acid. On the other hand, in the strain of which ywtD, ggt and gltA genes were all disrupted, not only productivity of poly-γ-glutamic acid was improved, but also poly-γ-glutamic acid was not degraded even when the culture time was extended. Further, a marked amount of poly-γ-glutamic acid could be produced and accumulated. These results revealed that the activity of poly-γ-glutamic acid decomposing enzyme encoded by the ywtD gene and the activity of γ-glutamyltranspeptidase encoded by the ggt gene greatly affected production of poly-γ-glutamic acid even in the poly-γ-glutamic acid producing strain of which productivity was improved by disruption of the gltA gene.

What is claimed is:

1. An endo-type poly-γ-glutamic acid decomposing enzyme having the following characteristics:
1) Substrate specificity: acting on poly-γ-glutamic acid having a molecular weight of 200 kDa or more to produce poly-γ-glutamic acid having a molecular weight of 10-50 kDa
2) Optimum pH: pH 5.0
3) pH stability: stable at pH 4.0-11.0 (treated at 4° C. for 16 hours)
4) Optimum temperature: around 45° C.
5) Temperature stability: stable up to 35° C. (treated at pH 7.0 for 60 minutes)
6) Effect of addition of metal ions and inhibitor (addition of 5 mM): activated by Ba²⁺ and Mn²⁺, and its activity is inhibited by Cu²⁺ and Ni²⁺, but not affected by addition of 5 mM EDTA.
7) Molecular weight: about 46 kDa (molecular weight measured by SDS-polyacrylamide gel electrophoresis or gel filtration).

2. The endo-type poly-γ-glutamic acid decomposing enzyme according to claim 1, which is a protein defined in the following (A) or (B):
(A) a protein having the amino acid sequence of SEQ ID NO: 2 shown in Sequence Listing;
(B) a protein having the amino acid sequence of SEQ ID NO: 2 shown in Sequence Listing including substitution, deletion, insertion or addition of one or several amino acids, and an endo-type poly-γ-glutamic acid decomposing enzyme activity.

3. A DNA coding for a protein defined in the following (A) or (B):
(A) a protein having the amino acid sequence of SEQ ID NO: 2 shown in Sequence Listing;
(B) a protein having the amino acid sequence of SEQ ID NO: 2 shown in Sequence Listing including substitution, deletion, insertion or addition of one or several amino acids, and an endo-type poly-γ-glutamic acid decomposing enzyme activity.

4. The DNA according to claim 3, which is defined in the following (a) or (b):
(a) a DNA which comprises the nucleotide sequence of the nucleotide numbers 41-1279 of SEQ ID NO: 1 shown in Sequence Listing;
(b) a DNA which is hybridizable with DNA having the nucleotide sequence of the nucleotide numbers 41-1279 of SEQ ID NO: 1 shown in Sequence Listing or a probe that can be prepared from the nucleotide sequence under a stringent condition, and codes for a protein having an endo-type poly-γ-glutamic acid decomposing enzyme activity.

5. The DNA according to claim 4, wherein the stringent condition is a condition that washing is performed at 60° C. with salt concentrations corresponding to 1×SSC and 0.1% SDS.

6. A microorganism belonging to the genus Bacillus, which has poly-γ-glutamic acid producing ability and is modified so that activity of the endo-type poly-γ-glutamic acid decomposing enzyme according to claim 1 should be reduced or eliminated.

7. The microorganism according to claim 6, which is modified so that the activity of the endo-type poly-γ-glutamic acid decomposing enzyme according to claim 1 or 2 should be reduced or eliminated by suppressing expression of a gene coding for the enzyme.

8. The microorganism according to claim 7, wherein expression of the gene coding for the endo-type poly-γ-glutamic acid decomposing enzyme according to claim 1 or 2 is suppressed by disrupting the gene.

9. The microorganism according to claim 8, wherein the gene coding for the endo-type poly-γ-glutamic acid decomposing enzyme according to claim 1 or 2 is disrupted by inclusion of substitution, deletion, insertion or addition of one or several nucleotides in the nucleotide sequence of the gene.

10. The microorganism according to claim 6, which is further modified so that γ-glutamyltranspeptidase activity should be reduced or eliminated.

11. The microorganism according to claim 6 or 10, which is further modified so that glutamate synthase activity should be reduced or eliminated.

12. A method for producing poly-γ-glutamic acid, which comprises culturing a microorganism according to any one of claims 6-11 in a liquid medium to produce and accumulate poly-γ-glutamic acid in a culture broth and collecting the poly-γ-glutamic acid.


SEQUENCE LISTING (6 sequences)

SEQ ID NO: 1 - DNA, 1285 bp, Bacillus subtilis
  CDS (41)..(1279)
ggatccgttaaaactgcaaaaagaggaggagataataaaagtgaacacactggcaaactggaagaagtttttgcttgtggcggttatcatttgttttttggttccaattatgacaaaagcggagattgcggaagctgatacatcatcagaattgattgtcagcgaagcaaaaaacctgcttggatatcagtataaatatggcggggaaacgccgaaagagggtttcgatccatcaggattgatacaatatgtgttcagtaaggctgatattcatctgccgagatctgtaaacgaccagtataaaatcggaacagctgtaaagccggaaaacctgaagccgggtgatattttgtttttcaagaaagagggaagcaacggctctgttccgacacatgacgccctttatatcggagacggccaaatggtacacagtacacagtcaaaaggggttatcatcaccaattacaaaaaaagcagctattggagcggaacttatatcggagcgagacgaatcgctgccgatccggcaacggctgatgttcctgtcgttcaggaggccgaaaaatatatcggtgtcccatatgtgtttggcggaagcacgccgtcagagggctttgattgctcggggcttgtgcaatatgtgtttcaacaggcactcggcatttatctaccgcgatcagccgaacagcagtgggcagtgggcgagaagatagcccctcagaacataaagcctggtgatgtcgtctatttcagcaatacgtataaaacgggaatttcacatgcaggcatttatgcgggcgcaggcaggttcatccaggcaagcaggtcagaaaaagtaaccatttcctatttgtcagaggattactggaaatcgaagatgacgggtattcgccgatttgacaacctgacaatcccgaaagaaaatccgattgtttccgaagcgacgctttatgtcggagaagtgccttacaaacagggcggagtaacacctgagacaggatttgatacagctggatttgtccaatatgtataccagaaagcagccggtatttccctgcctcgatacgcaacaagccagtacaatgccggaactaagattaagaaggcggacctgaagccgggagacattgtgttctttcaatcaacaagcttaaatccctccatctatatcggaaacggacaagttgttcatgtcacattatcaaacggcgtgaccatcaccaatatgaacacgagcacatattggaaggataaatacgcaggaagtatacgggtgcaactcgag

SEQ ID NO: 2 - protein, 413 aa, Bacillus subtilis
VNTLANWKKFLLVAVIICFLVPIMTKAEIAEADTSSELIVSEAKNLLGYQYKYGGETPKEGFDPSGLIQYVFSKADIHLPRSVNDQYKIGTAVKPENLKPGDILFFKKEGSNGSVPTHDALYIGDGQMVHSTQSKGVIITNYKKSSYWSGTYIGARRIAADPATADVPVVQEAEKYIGVPYVFGGSTPSEGFDCSGLVQYVFQQALGIYLPRSAEQQWAVGEKIAPQNIKPGDVVYFSNTYKTGISHAGIYAGAGRFIQASRSEKVTISYLSEDYWKSKMTGIRRFDNLTIPKENPIVSEATLYVGEVPYKQGGVTPETGFDTAGFVQYVYQKAAGISLPRYATSQYNAGTKIKKADLKPGDIVFFQSTSLNPSIYIGNGQVVHVTLSNGVTITNMNTSTYWKDKYAGSIRVQ

SEQ ID NO: 3 - DNA, 26 bp, ARTIFICIAL SEQUENCE
  SYNTHETIC DNA
ggatccgttaaaactgcaaaaagagg

SEQ ID NO: 4 - DNA, 26 bp, ARTIFICIAL SEQUENCE
  SYNTHETIC DNA
tttctcgagttgcacccgtatacttc

SEQ ID NO: 5 - DNA, 22 bp, ARTIFICIAL SEQUENCE
  SYNTHETIC DNA
cttggatggagaactgtacctg

SEQ ID NO: 6 - DNA, 20 bp, ARTIFICIAL SEQUENCE
  SYNTHETIC DNA
ggcgctgaaattaggtgctg